(19) United States
(12) Patent Application Publication
(10) Pub. No.: US 2018/0007322 A1
(43) Pub. Date: Jan. 4, 2018

(54) ENDOSCOPE SYSTEM

(71) Applicant: OYLMPUS CORPORATION, Tokyo (JP)

(72) Inventor: Hiroyuki KOJO, Hachioji-shi (JP)

(73) Assignee: OLYMPUS CORPORATION, Tokyo (JP)

(21) Appl. No.: 15/200,446

(22) Filed: Jul. 1, 2016

(51) Int. Cl. H04N 7/18; G08B 6/00; A61B 1/00; H04N 5/225

(52) U.S. Cl. CPC H04N 7/185; A61B 1/00055; G08B 6/00; A61B 1/00179; A61B 1/00009; A61B 1/00045; A61B 1/00039; H04N 2005/2255

(57) ABSTRACT

An endoscope system includes, an inserting section having an insertion axis, an imaging section which takes an image of a specimen on a lateral side to the insertion axis, an image generating section which generates a picture image of the specimen on the basis of an imaging signal output by the imaging section, an operating section which is provided on a proximal end side of the inserting section, and operated by an operator, a gravity direction indicating section which is provided on the lateral side of the operating section, and used by the operator to indicate a gravity direction, and a processing section which rotates the picture image of the specimen in correspondence with an indication of the gravity direction indicating section.

### 1. FIELD OF THE INVENTION

The present invention relates to an endoscope system which can visually recognize the inside of a hole of a specimen.

### 2. DESCRIPTION OF THE RELATED ART

For example, Jpn. Pat. Appln. KOKAI Publication No. 2003-290119 discloses a medial endoscope which observes an organ or the like in a body cavity and diagnoses an affected part in detail by inserting an elongated inserting section into the body cavity.

### BRIEF SUMMARY OF THE INVENTION

An endoscope system includes, an inserting section having an insertion axis, an imaging section which takes an image of a specimen on a lateral side to the insertion axis, an image generating section which generates a picture image of the specimen on the basis of an imaging signal output by the imaging section, an operating section which is provided on a proximal end side of the inserting section, and operated by an operator, a gravity direction indicating section which is provided on the lateral side of the operating section, and used by the operator to indicate a gravity direction, and a processing section which rotates the picture image of the specimen in correspondence with an indication of the gravity direction indicating section.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view showing an entire configuration of an endoscope system according to an embodiment;

FIG. 2 is a top view showing an insertion apparatus of the endoscope system depicted in FIG. 1 from above;

FIG. 3 is a schematic view schematically showing an endoscope inserting section and an endoscope imaging section of the insertion apparatus of the endoscope system depicted in FIG. 1;

FIG. 4 is a schematic view schematically showing an endoscope inserting section and an endoscope imaging section according to a modification of the insertion apparatus of the endoscope system depicted in FIG. 1;

FIG. 5 is a schematic view schematically showing that a lower direction of a picture image in a display section is displaced to a gravity direction when a twisting operation to rotate an operating section around an imaging axis is performed to the insertion apparatus of the endoscope system depicted in FIG. 1;

FIG. 6 is a schematic view schematically showing that the lower direction of the picture image in the display section can be aligned with the gravity direction by aligning an indicator of a rotary operating section with the gravity direction in the endoscope system depicted in FIG. 5;

FIG. 7 is a schematic view schematically showing a step of matching the lower direction of the picture image displayed in the displays section with the gravity direction in the display section of the endoscope system depicted in FIG. 1;

FIG. 8 is a schematic view schematically showing that the lower direction of the picture image in the display section is displaced to the gravity direction when the twisting operation is performed to the operating section in the insertion apparatus according to a first modification of the endoscope system according to the embodiment;

FIG. 9 is a schematic view showing the rotary operating section and a gravity indicator in the insertion apparatus of the endoscope system depicted in FIG. 8;

FIG. 10 is a schematic view showing a step of matching the indicator of the rotary operating section with the gravity direction indicated by the gravity indicator in the rotary operating section and the gravity indicator depicted in FIG. 9;

FIG. 11 is a schematic view showing the rotary operating section and the gravity indicator in the insertion apparatus according to a second modification of the endoscope system according to the embodiment; and

FIG. 12 is a schematic view showing a step of matching the indicator of the rotary operating section with a direction opposite to the gravity direction indicated by the gravity indicator in the rotary operating section and the gravity indicator depicted in FIG. 11.

### DETAILED DESCRIPTION OF THE INVENTION

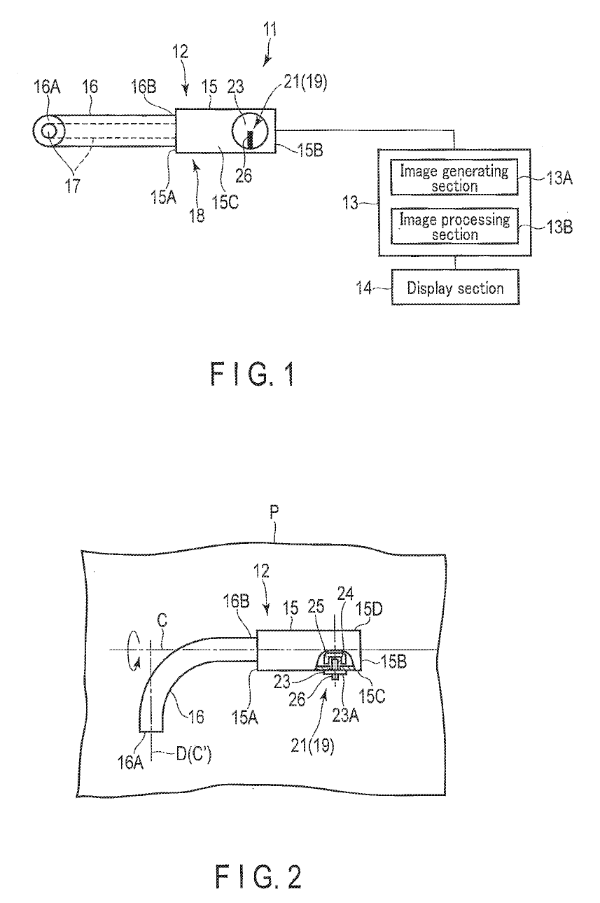

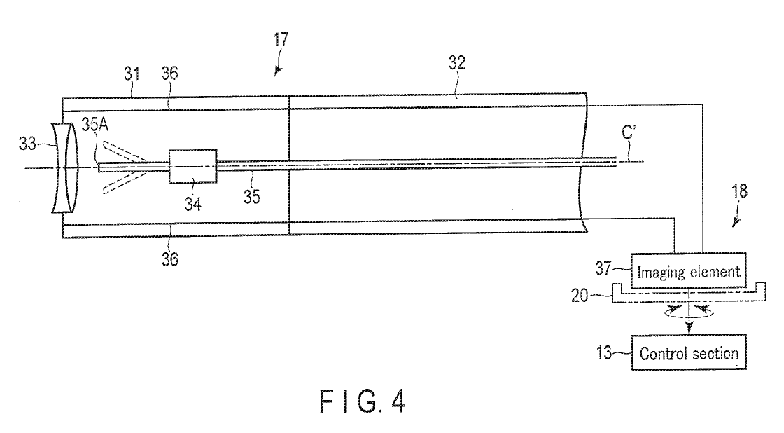

An embodiment will now be described hereinafter with reference to FIG. 1 to FIG. 7.

As shown in FIG. 1, an endoscope system 11 according to this embodiment has an insertion apparatus 12 which is used while being inserted into a hole of a specimen (e.g., a nasal sinus), a control section 13 which is connected to the insertion apparatus 12, and a display section 14 which is connected to the control section 13. The insertion apparatus 12 is provided separately from the display section 14. The display section 14 is constituted of a general liquid crystal monitor.

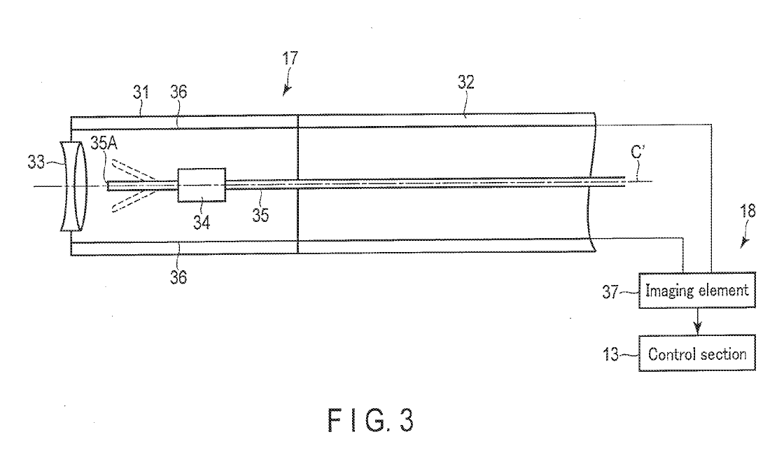

As shown in FIG. 1 and FIG. 3, the insertion apparatus 12 has an operating section 15 which forms an outer envelope and is operated by an operator, a tubular inserting section 16 which protrudes from a distal end portion 15A of the operating section 15, an endoscope inserting section 17 which is inserted into the inserting section 16 and the operating section 15, an endoscope imaging section 18 (an imaging section) provided inside the operating section 15, and a gravity direction indicating section 19 provided on a lateral side 15C of the operating section 15. In this embodiment, although the endoscope inserting section 17 and the endoscope imaging section 18 are separated from each other, they may be integrated to constitute an endoscope. In this embodiment, the gravity direction indicating section 19 is used by the operator to indicate a gravity direction. The gravity direction indicating section 19 is constituted of, e.g., a later-described rotary operating section 21. The gravity direction indicating section 19 may be provided on an opposite surface 15D (a surface facing the lateral side 15C (see FIG. 2)) of the lateral side 15C.

The operating section 15 constitutes a portion gripped by a user's hand. The operating section 15 is provided on a proximal end side of the inserting section 16. The operating section 15 has a distal end portion 15A and a proximal end portion 15B. As shown in FIG. 2, a central axis C direction (an axial direction) of the operating section 15 is defined by the distal end portion 15A and the proximal end portion 15B. The central axis C (a longitudinal axis) of the operating section 15 coincides with a central axis of the endoscope inserting section 17 on the proximal end side inserted into the operating section 15 and a central axis of the endoscope imaging section 18. However, the central axis C of the operating section 15 may deviate from the central axes of the endoscope inserting section 17 and the endoscope imaging section 18.

As shown in FIG. 1, the inserting section 16 has a cylindrical shape, and can guide the endoscope inserting section 17, which is inserted therein, along an inner wall. As shown in FIG. 2, the inserting section 16 has an insertion axis C, a distal end 16A, and a proximal end 16B connected to the operating section 15 on the opposite side of the distal end 16A. The insertion axis C coincides with the central axis C of the operating section 15. A direction D along which the inserting section 16 (the distal end 16A of the inserting section 16) protrudes is different from the central axis C direction C of the operating section 15. The inserting section 16 bends from a direction parallel to the central axis C of the operating section 15 toward a protruding direction D different from the central axis C direction, and can be easily inserted into a nasal sinus of a patient (a specimen). The protruding direction D crosses (is substantially orthogonal to) the central axis C. The protruding direction D coincides with a central axis C′ of the endoscope inserting section 17.

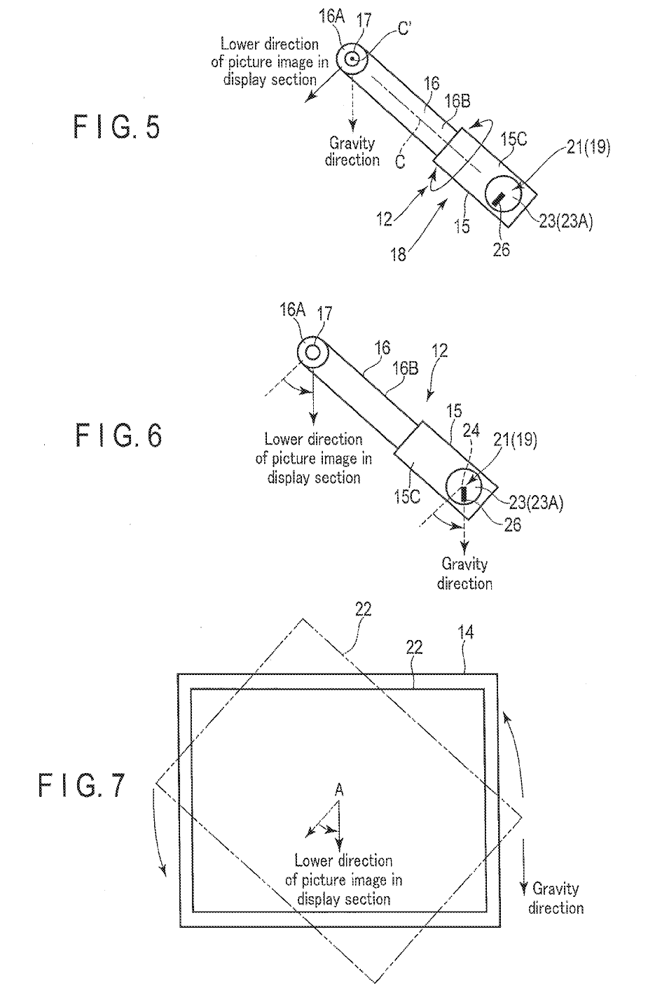

As shown in FIG. 2, the inserting section 16 is rotatable to the operating section 15, and can rotate around the central axis C of the operating section 15. A rotation knob which allows the inserting section 16 to rotate around the central axis C to the operating section 15 may be provided to the operating section 15. An advancing/retreating mechanism which advances or retreats the endoscope inserting section 17 in the central axis C direction to the inserting section 16 may be provided to the operating section 15. That is, in a medical examination, when a physician who is a user changes positions of the inserting section 16 and the endoscope inserting section 17 by using the rotation knob, the advancing/retreating mechanism, and others in a state where the inserting section 16 is inserted in a hole of a specimen (a sinus of a paranasal cavity), a desired picture image 22 of the inside of the hole can be acquired. Further, as shown in FIG. 5, the user can acquire a desired picture image 22 by performing an operation to rotate the operating section 15 and the inserting section 16 around the central axis C (which will be referred to as a twisting operation hereinafter) without rotating the inserting section 16 to the operating section 15.

As shown in FIG. 2, the central axis C direction of the operating section 15 and the protruding direction D of the inserting section 16 define one plane P including these directions.

The rotary operating section 21 is provided to be rotatable to the operating section 15. When a user operates the rotary operating section 21, he/she can change a display angle of the picture image 22 displayed in the display section 14. As shown in FIG. 1 and FIG. 2, the rotary operating section 21 has, e.g., a discoid dial section 23, a rotary shaft 24 which supports the dial section 23 and faces the lateral side 15C (extends in a direction crossing the lateral side 15C), and a potentiometer 25 which detects a rotation angle of the rotary shaft 24. As shown in FIG. 6, the rotary operating section 21 can rotate in a counterclockwise direction (a direction indicated by an arrow in the drawing) around the rotary shaft 24 and a clockwise direction opposite to the former direction as seen from a direction facing the rotary operating section 21. As shown in FIG. 2, the dial section 23 has a flat surface 23A, and an indicator 26 (an indicating convex portion) which protrudes from the surface 23a toward a direction opposite to a direction along which the rotary shaft 24 is provided.

Since the indicator 26 protrudes from the surface 23A, the user can be aware of an indicated direction of the indicator 26 by a feeling when he/she touches with his/her finger, i.e., a sense of touch without being dependent on a sense of sight. Further, a shape of the indicator 26 is an example, and the indicator 26 may have a different shape as long as the user can be aware of the indicated direction by the sense of touch. That is, the indicator 26 may be formed by recessing the flat surface 23A.

As shown in FIG. 2, the rotary shaft 24 extends within the one plane P, and extends in a direction parallel to the protruding direction D of the inserting section 16. Here, the direction parallel to the protruding direction D is a concept including the direction parallel to the protruding direction D and a nearly parallel direction which is inclined several degrees to the protruding direction D. The rotary shaft 24 may be displaced from this one plane P by a very small angle or a very short distance. That is, this rotary shaft 24 may be arranged on another plane which is parallel to the one plane P and apart from the one plane P by a very short distance, or may be arranged on another plane which includes the central axis C and is rotated around the central axis C at a very small angle from the one plane P. In this manner, the rotary shaft 24 may be substantially arranged on the one plane P like a case where it is arranged on the one plane P as well as a case where it is arranged on another plane slightly deviating from the one plane P as described above.

When the dial section 23 is operated by the user, a rotation angle of the rotary shaft 24 which rotates together with the dial section 23 is detected by the potentiometer 25.

As shown in FIG. 3, the endoscope inserting section 17 is configured to have flexibility as a whole, and can bend along a shape of the inserting section 16 when it is inserted into the inserting section 16. The endoscope inserting section 17 and the endoscope imaging section 18 are constituted as a so-called scanning endoscope. The central axis C′ of the endoscope inserting section 17 is defined in a longitudinal direction thereof. The endoscope inserting section 17 has a distal end constituting section 31 placed on a distal end side of the central axis C′ direction, a flexible tube 32 provided on a proximal end side of the distal end constituting section 31 in the central axis C′ direction, an illumination window 33, an actuator 34, an illumination fiber 35, and light receiving fibers 36. The illumination fiber 35 is optically connected to a light source provided to be adjacent to the control section 13. The light receiving fibers 36 are optically connected to an imaging element 37.

The endoscope imaging section 18 has the imaging element 37 formed of a CCD, CMOS, or the like. The endoscope imaging section 18 can acquire an image provided at the distal end of the inserting section 16, and can take an image of a specimen on a lateral side (the direction parallel to the protruding direction D) to the insertion axis C through the endoscope inserting section 17. More specifically, the imaging element 37 can convert lights from the light receiving fibers 36 into an electrical signal and supplies it to the control section 13.

The actuator 34 is electrically connected to the control section 13. The actuator 34 is, for example, spirally rocked by the control section 13. Therefore, a distal end 35A of the illumination fiber 35 is spirally rocked in accordance with the operation of the actuator 34. Thus, a surface of the specimen is spirally scanned by illumination light from the illumination fiber 35 through the distal end 35A of the illumination fiber 35 and the illumination window 33. The light receiving fibers 36 receive return light from the specimen, and guide the light to the imaging element 37. The imaging element 37 supplies a picture image to the control section 13 by using the lights received by the light receiving fibers 36 as electrical signal. The control section 13 converts the electrical signal into a picture image, appropriately executes image processing, and displays it in the display section 14.

The control section 13 shown in FIG. 1 is constituted of, e.g., a general computer and software which is installed in this computer and performs various kinds of control over the insertion apparatus 12. The control section 13 has an image generating section 13A which generates a picture image to be displayed in the display section, and an image processing section 13B which rotates a picture image of the specimen in correspondence with an indication of the gravity direction indicating section. The image generating section 13A generates the picture image of the specimen on the basis of an imaging signal output from the endoscope imaging section 18 (the imaging section).

The control section 13 can perform, e.g., the following control to each section in the insertion apparatus 12. The control section 13 can adjust the number of revolutions and others of the actuator 34 by controlling the actuator 34 which rocks the illumination fiber 35. The control section 13 can adjust a light quantity which is supplied to the illumination fiber 35 by controlling the light source. The image processing section 13B can perform image processing to rotate the picture image 22 of the specimen around a picture image center A in correspondence with an indication to the gravity direction indicating section 19 (a rotating operation of an operator to the rotary operating section 21). The image processing section 13B is an example of a processing section which rotates the picture image 22 of the specimen in correspondence with an indication of the gravity direction indicating section 19.

It is to be noted that the endoscope system 11 may include a rotary processing section 20 which mechanically rotates the endoscope imaging section 18 (the imaging element 37) in place of the image processing section 13B as shown in FIG. 5. The rotary processing section 20 is formed into a rotary table, and can rotate the endoscope imaging section 18 (the imaging element 37) mounted on this rotary table. In this modification, the picture image 22 actually displayed in the display section 14 can be rotated by rotating the endoscope imaging section 18 (the imaging element 37) with the use of the rotary processing section 20 in place of rotating the picture image by the image processing. In this modification, the rotary processing section 20 is an example of a processing section which rotates the picture image 22 of the specimen in correspondence with an indication of the gravity direction indicating section 19.

The control section 13 can make a picture image of an electrical signal corresponding to an image acquired by the imaging element 37 of the insertion apparatus 12, and display it as a picture image in the display section 14. The control section 13 can change a display angle of the picture image 22 displayed in the display section 14 on the basis of an operation given to the rotary operating section 21 from the user (see FIG. 7). That is, the control section 13 displays this picture image in the display section 14 so that a lower direction of the picture image 22 displayed in the display section 14 corresponds to an indicated direction of the indicator 26 provided through the potentiometer 25. Thus, the control section 13 can rotate the picture image 22 of the display section 14 in the same direction as the rotating direction of the rotary operating section 21. The control section 13 can rotate the picture image 22 in the display section 14 so that a rotation angle of the rotary operating section 21 can substantially coincide with a rotation angle of the picture image 22.

A function of the endoscope system 11 according to this embodiment will now be described with reference to FIG. 5 to FIG. 7.

In a medical examination, a physician who is the user can insert the inserting section 16 into a sinus of a nasal cavity or a paranasal cavity of a patient (the specimen) to observe the sinus. In the medical examination, even if the user performs, e.g., an operation to rotate the inserting section 16 around the central axis C of the operating section 15, an installation angle of the endoscope imaging section 18 present in the operating section 15 remains the same, and hence the lower direction of the picture image 22 displayed in the display section 14 is not displaced to the gravity direction of the operating section 15.

On the other hand, for example, as shown in FIG. 5, when the user performs a twisting operation to rotate the operating section 15 around the central axis C, since the installation angle of the endoscope imaging section 18 changes, the lower direction of the picture image 22 in the display section 14 is displaced from the gravity direction. In this case, as shown in FIG. 6, when the user rotates the rotary operating section 21 in the counterclockwise direction to align the indicator 26 with the gravity direction, the lower direction of the picture image 22 displayed in the display section 14 can be aligned with the gravity direction. That is, the picture image in the display section 14 is rotated in the counterclockwise direction around the picture image center A as shown in FIG. 7 and as indicated by an arrow in this drawing by the image processing in the image processing section 13B of the control section 13. At this time, since the indicator 26 can be recognized by the user's sense of touch, the user can perform the rotating operation of the picture image 22 without taking his/her eyes from the display section 14.

According to the embodiment, the following can be said. That is, endoscope system 11 has the inserting section 16 having the insertion axis C, the imaging section 18 which takes an image of the specimen on the lateral side to the insertion axis C, the image generating section 13A which generates a picture image of the specimen on the basis of an imaging signal output from the imaging section 18, the operating section 15 which is provided on the proximal end side of the inserting section 16 and operated by the operator, the gravity direction indicating section 19 which is provided on the lateral side of the operating section 15 and used by the operator to indicate a gravity direction, and the processing section which rotates the picture image of the specimen in correspondence with an indication of the gravity direction indicating section 19.

When an imaging direction is different from the insertion axis C, a physician who is the user tends to lose sight of the gravity direction in the picture image 22 displayed in the display section 14. In particular, a viewing field is often dark in a narrow hole of the specimen and the user tends to lose sight. According to the above-described configuration, for example, when the user performs an operation of displacing the lower direction of the picture image 22 from the gravity direction, an operation of rotating the picture image 22 can be performed by an indication using the gravity direction indicating section 19. Consequently, even if the physician who is a user loses sight of the direction during an examination using the endoscope system 11, it is possible to rapidly return to a state where the direction can be recognized. Consequently, a position of an affected part of a patient can be accurately grasped, and an appropriate examination can be carried out.

The endoscope system 11 has the display section 14, the gravity direction indicating section 19 is the rotary operating section 21, the rotary operating section 21 can turn around the rotary shaft 24 facing the lateral side, and a display angle of the picture image 22 acquired from the image and displayed in the display section 14 can be changed when the rotary operating section 21 is rotated around the rotary shaft 24. According to this configuration, the operation of rotating the picture image 22 can be intuitively performed by the rotating operation using the rotary operating section 21.

The control section 13 rotates the picture image 22 in the same direction as the direction in which the rotary shaft 24 is rotated. According to this configuration, the picture image 22 can be rotated without bringing a feeling of strangeness to the user, and the intuitive operation by the user can be realized.

The control section 13 rotates the picture image so that a rotation angle of the rotary operating section 21 substantially coincides with a rotation angle of the picture image 22. According to this configuration, the intuitive operation by the user can be realized, convenience for the user can be improved.

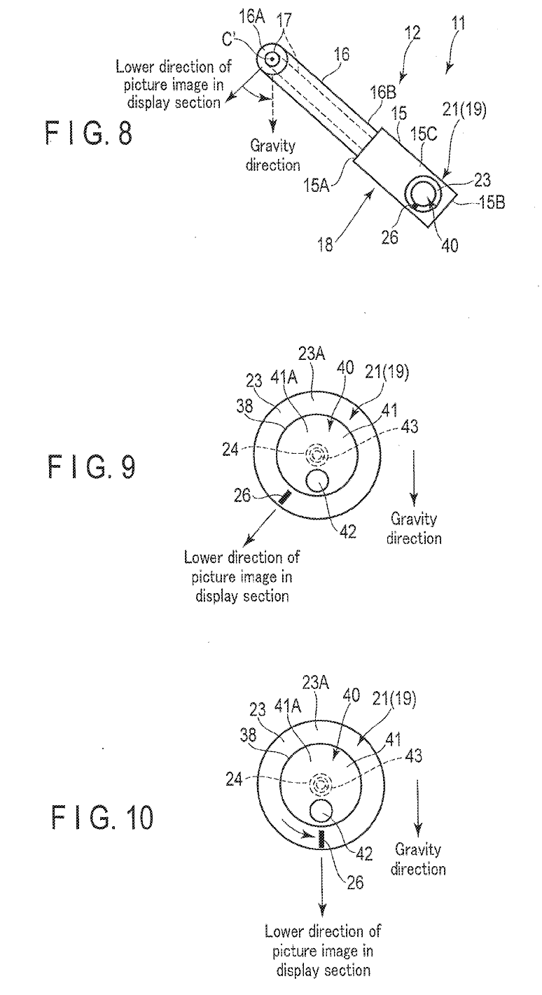

A first modification of this embodiment will now be described with reference to FIG. 8 to FIG. 10. In the first modification described below, parts different from the foregoing embodiment will be mainly described, and a description of parts common to the first embodiment will be omitted.

As shown in FIG. 8, in this modification, the insertion apparatus 12 has the operating section 15, the inserting section 16, the endoscope inserting section 17, the endoscope imaging section 18 (the imaging section), the rotary operating section 21, and a gravity indicator 40 which indicates a gravity direction.

The rotary operating section 21 has, e.g., the discoid dial section 23, the rotary shaft 24 which supports the dial section 23, and the potentiometer 25 which detects a rotation angle of the rotary shaft 24 (see FIG. 2). In this modification, as shown in FIG. 9, the rotary shaft 24 is formed into a hollow shape so that a later-described second rotary shaft 43 is inserted therein. The dial section 23 has the flat surface 23A, a concave portion 38 which is provided at a central portion of the surface 23A and circularly recessed so that the gravity indicator 40 can be accommodated, and the indicator 26 which protrudes from the surface 23A. The control section 13 displays the picture image 22 in the display section 14 so that a lower direction of the picture image 22 displayed in the display section 14 coincides with an indicated direction of the indicator 26 provided through the potentiometer 25.

Furthermore, a shape of the indicator 26 is an example, and the indicator 26 may have a different shape as long as the user can be aware of an indicated direction by a sense of touch. That is, the indicator 26 may be formed by recessing the flat surface 23A.

As shown in FIG. 8 and FIG. 9, the gravity indicator 40 is accommodated in the concave portion 38 of the rotary operating section 21, and provided to be rotatable to the operating section 15 and the rotary operating section 21. The gravity indicator 40 is provided near the rotary operating section 21 or to be adjacent to the rotary operating section 21. The gravity indicator 40 has a discoid support section 41, a weight 42 disposed to be eccentric to a central portion of the support section 41, and a second rotary shaft 43 which rotatably supports the support section 41 to the rotary operating section 21. The second rotary shaft 43 is arranged in an inner hollow portion of the rotary shaft 24 to be concentric with the rotary shaft 24. Thus, the gravity indicator 40 is rotatable to the rotary operating section 21. Furthermore, the weight 42 is always placed on a gravity direction (lower direction) side to the central portion of the support section 41. Thus, the gravity indicator 40 can indicate a gravity direction by using the weight 42.

A support surface 41A of the support section 41 is flat. The weight 42 is disposed to the support section 41 to protrude from the support surface 41A of the support section 41. Thus, the user can always recognize the gravity direction by not only a sense of sight but also a feeling (a sense of touch) when he/she touches the gravity indicator 40 with his/her finger or the like.

In this modification, since the weight 42 of the gravity indicator 40 protrudes from the support surface 41A and the indicator 26 of the rotary operating section 21 protrudes from the surface 23A, the user can be aware of an indicated direction of the gravity indicator 40 and an indicated direction of the indicator 26 by a feeling when he/she touches with his/her finger, i.e., a sense of touch without being dependent on a sense of sight. Further, arrangement and a shape of the weight 42 are just examples, and the arrangement and the shape of the weight 42 may be substituted by different ones as long as the user can be aware of an indicated direction by the sense of touch. That is, the weight 42 may be accommodated in the support section 41 and arranged in a recess which is recessed from the flat support surface 41A. For example, with such a configuration, the user may be aware of the recess where the weight 42 is accommodated by a feeling (a sense of touch) when the user touches the recess with his/her finger or the like.

A function of the endoscope system 11 according to this embodiment will now be described with reference to FIG. 8 to FIG. 10.

When a physician who is the user performs, e.g., a twisting operation of rotating the operating section 15 around the central axis C like the foregoing embodiment, as shown in FIG. 8, an installation angle of the endoscope imaging section 18 changes, and hence a lower direction of the picture image 22 in the display section 14 is displaced from a gravity direction. In this case, as shown in FIG. 9, the weight 42 of the gravity indicator 40 is placed on the gravity direction (the lower direction) side to the central portion (a portion corresponding to the second rotary shaft 43) of the gravity indicator 40. On the other hand, it is assumed that the indicator 26 of the rotary operating section 21 indicates a left obliquely downward direction in FIG. 9. In this case, the user rotates the rotary operating section 21 in the counterclockwise direction to be matched with a position of the weight 42 as shown in FIG. 10 while visually confirming a displacement between the weight 42 and the indicator 26, thereby matching the lower direction of the picture image 22 displayed in the display section 14 with the gravity direction.

Alternatively, the user can also adjust an angle of the picture image 22 displayed in the display section 14 while watching the picture image 22 in the display section 14 without seeing the rotary operating section 21 and the gravity indicator 40. In this case, the user can recognize the displacement of the lower direction of the picture image 22 of the display section 14 from the gravity direction by a feeling (a sense of touch) when he/she touches the indicator 26 and the weight 42 with his/her finger, and can match the lower direction of the picture image 22 displayed in the display section 14 with the gravity direction by rotating the rotary operating section 21 in the counterclockwise direction to match the indicator 26 with a position of the weight 42.

According to this modification, the following can be said. That is, the operating section 15 has the gravity indicator 40, and the gravity indicator 40 is provided near the rotary operating section 21 and indicates the gravity direction. According to this configuration, the user can easily recognize the gravity direction, and rapidly change a display angle of the picture image 22 displayed in the display section 14 to an easy-to-see angle (e.g., an angle at which the lower direction of the picture image 22 coincides with the gravity direction).

The rotary operating section 21 has the indicator 26, and the indicator 26 corresponds to the lower direction of the picture image 22. According to this configuration, for example, when the indicator 26 is matched with an indicated direction of the gravity indicator 40, the lower direction of the picture image 22 can be easily matched with the gravity direction. Consequently, even when visual contact with a direction is lost during an examination using the endoscope system 11, it is possible to rapidly return to a state where the direction can be recognized. As a result, the user can easily recognize a position of an affected part, thereby considerably improving the convenience for a physician who is the user.

An indicated direction of the indicator 26 and an indicated direction of the gravity indicator 40 can be recognized by the user's sense of touch. According to this configuration, the user can match the lower direction of the image in the display section 14 with the gravity direction by using the sense of touch alone without taking his/her eyes from the display section 14. Consequently, it is possible to considerably improve the convenience for the physician who is the user.

The gravity indicator 40 has the weight 42. According to this configuration, the structure of the gravity indicator 40 can be simplified, and manufacturing costs of the endoscope system 11 can be reduced.

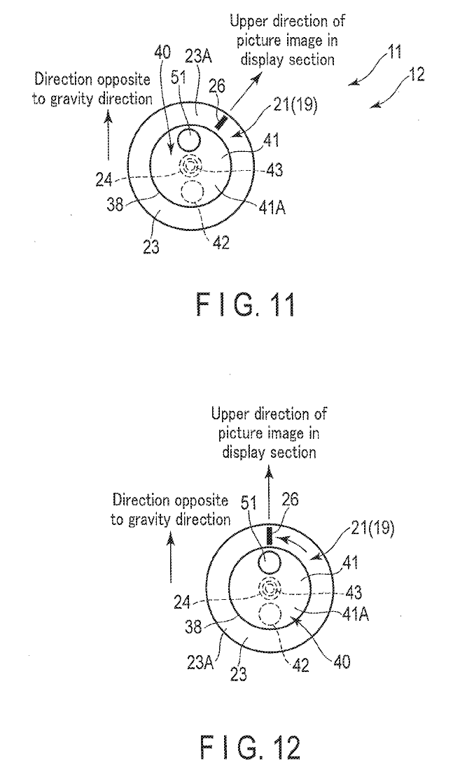

A second modification of this embodiment will now be described hereinafter with reference to FIG. 11 and FIG. 12. In the second modification described below, parts different from the foregoing embodiment and the first modification will be mainly described, and a description on parts common to the foregoing embodiment and the first modification will be omitted.

In this modification, the insertion apparatus 12 has the operating section 15, the inserting section 16, the endoscope inserting section 17, the endoscope imaging section 18 (the imaging section), the rotary operating section 21, and the gravity indicator 40 which indicates a direction opposite to the gravity direction.

The rotary operating section 21 has, e.g., the discoid dial section 23, the rotary shaft 24 which supports the dial section 23, and the potentiometer 25 which detects a rotation angle of the rotary shaft 24. In this modification, the rotary shaft 24 is formed into a hollow shape. The dial section 23 has the flat surface 23A, the concave portion 38 which is provided at a central portion of the surface 23A and circularly recessed so that the gravity indicator 40 can be accommodated, and the indicator 26 which protrudes from the surface 23A. The control section 13 displays a picture image 22 in the display section 14 so that an upper direction of the picture image 22 displayed in the display section 14 corresponds to an indicated direction of the indicator 26 provided through the potentiometer 25.

Furthermore, a shape of the indicator 26 is an example, and the indicator 26 may have a different shape as long as the user can be aware of an indicated direction by a sense of touch. That is, the indicator 26 may be formed by recessing the flat surface 23A.

The gravity indicator 40 is provided in the concave portion 38 of the rotary operating section 21 to be rotatable to the operating section 15 and the rotary operating section 21. The gravity indicator 40 is provided near the rotary operating section 21 or to be adjacent to the rotary operating section 21. The gravity indicator 40 has the discoid support section 41, the weight 42 disposed to be eccentric to a central portion of the support section 41, the second rotary shaft 43 which rotatably supports the support section 41 to the rotary operating section 21, and a protruding section 51 which is provided on the opposite side of the weight 42 to the central portion of the support section 41 (a portion corresponding to the second rotary shaft 43). The second rotary shaft 43 is arranged in an inner hollow portion of the rotary shaft 24 to be concentric with the rotary shaft 24. Thus, the gravity indicator 40 is rotatable to the rotary operating section 21. The weight 42 is fixed in, e.g., a recess formed on a support back surface on the opposite side of the support surface 41A of the support section 41. The protruding section 51 is always placed on an opposite direction (the upper direction) side of the gravity direction (the lower direction) to the central portion. Thus, the gravity indicator 40 can indicate the opposite direction (an upper side in a perpendicular direction) of the gravity direction by the protruding section 51.

The support surface 41A of the support section 41 is flat. The protruding section 51 is disposed to the support section 41 to protrude from the support surface 41A of the support section 41. Thus, the user can always recognize the direction opposite to the gravity direction by not only a sense of sight but also a feeling (a sense of touch) when he/she touches the gravity indicator 40 with his/her finger or the like.

In this modification, since the protruding section 51 of the gravity indicator 40 protrudes from the support surface 41A and the indicator 26 of the rotary operating section 21 protrudes from the surface 23A, the user can be aware of an indicated direction of the gravity indicator 40 and an indicated direction of the indicator 26 by a feeling when he/she touches with his/her finger, i.e., a sense of touch without being dependent on a sense of sight. Furthermore, a shape of the protruding section 51 is an example, and the protruding section 51 may have a different shape as long as the user can be aware of the indicated directions by the sense of touch.

A function of the endoscope system 11 according to this embodiment will now be described with reference to FIG. 11 and FIG. 12.

When a physician who is the user performs a twisting operation to rotate the operating section 15 around the central axis C like the foregoing embodiment, since an installation angle of the endoscope imaging section 18 changes, the lower direction of the picture image 22 in the display section 14 is displaced from the gravity direction. This state can be rephrased as that an upper direction of the picture image 22 in the display section 14 is displaced from the direction opposite to the gravity direction,

In this case, as shown in FIG. 11, the weight 42 of the gravity indicator 40 is placed on the gravity direction (the lower direction) side to the central portion of the gravity indicator 40 (a portion corresponding to the second rotary shaft 43), and the protruding portion 51 of the gravity indicator 40 is placed on the opposite direction (the upper direction) side of the gravity direction to the central portion. On the other hand, it is assumed that the indicator 26 of the rotary operating section 21 indicates a right obliquely upward direction in FIG. 11. In this case, when the user rotates the rotary operating section 21 in the counterclockwise direction to match the indicator 26 with the protruding section 51 as shown in FIG. 12 by visually confirming a displacement between the protruding section 51 and the indicator 26, the upper direction of the picture image 22 displayed in the display section 14 can be matched with the direction opposite to the gravity direction.

Alternatively, the user can adjust an angle of the picture image 22 displayed in the display section 14 while watching the picture image 22 in the display section 14 without seeing the rotary operating section 21 and the gravity indicator 40. In this case, the user can recognize a displacement between the upper direction of the picture image 22 in the display section 14 and the direction opposite to the gravity direction by the feeling (the sense of touch) when he/she touches the indicator 26 and the protruding section 51 with his/her finger, and can match the upper direction of the picture image 22 displayed in the display section 14 with the direction opposite to the gravity direction by rotating the rotary operating section 21 in the counterclockwise direction to set the indicator 26 to a position of the protruding section 51.

According to this modification, the operating section 15 has the gravity indicator 40, and the gravity indicator 40 is provided near the rotary operating section 21 and indicates the direction opposite to the gravity direction. According to this configuration, like the first modification, a physician who is the user can easily recognize the gravity direction, and rapidly change a display angle of the picture image 22 displayed in the display section 14 to an easy-to-see angle (e.g., an angle at which the upper direction of the picture image 22 coincides with the direction opposite to the gravity direction).

The rotary operating section 21 has the indicator 26, and the indicator 26 corresponds to the upper direction of the picture image 22. According to this configuration, for example, when the indicator 26 is matched with an indicated direction of the gravity indicator 40, the upper direction of the picture image 22 can be easily matched with the direction opposite to the gravity direction. Consequently, the user can readily recognize a position of an affected part, and convenience for a physician who is the user can be greatly improved.

The indicated direction of the indicator 26 and the indicated direction of the gravity indicator 40 can be recognized by the user's sense of touch. According to this configuration, the user can match the lower direction of the picture image 22 in the display section 14 with the gravity direction by the sense of touch alone. Consequently, the convenience for a physician who is the user can be considerably improved.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. An endoscope system comprising:
an inserting section having an insertion axis;
an imaging section which takes an image of a specimen on a lateral side to the insertion axis;
an image generating section which generates a picture image of the specimen on the basis of an imaging signal output by the imaging section;
an operating section which is provided on a proximal end side of the inserting section, and operated by an operator;
a gravity direction indicating section which is provided on the lateral side of the operating section, and used by the operator to indicate a gravity direction; and
a processing section which rotates the picture image of the specimen in correspondence with an indication of the gravity direction indicating section.

2. The system according to claim 1, comprising a display section,
wherein the gravity direction indicating section is a rotary operating section, and the rotary operating section is turnable around a rotary shaft facing the lateral side and configured to change a display angle of a picture image, which is obtained from the image and displayed in the display section, when it is rotated around the rotary shaft.

3. The system according to claim 2,
wherein the processing section rotates the picture image in the same direction as a direction along which the rotary operating section is rotated.

4. The system according to claim 2,
wherein the processing section rotates the picture image so that a rotation angle of the rotary operating section substantially coincides with a rotation angle of the picture image.

5. The system according to claim 2,
wherein the operating section has a gravity indicator, and the gravity indicator is provided near the rotary operating section and indicates the gravity direction.

6. The system according to claim 5,
wherein the rotary operating section has an indicator, and the indicator corresponds to a lower direction of the picture image.

7. The system according to claim 6,
wherein an indicated direction of the indicator and an indicated direction of the gravity indicator are recognizable by a user's sense of touch.

8. The system according to claim 7,
wherein the gravity indicator has a weight.

9. The system according to claim 2,
wherein the operating section has a gravity indicator, and the gravity indicator is provided near the rotary operating section and indicates a direction opposite to the gravity direction.

10. The system according to claim 9,
wherein the rotary operating section has an indicator, and the indicator corresponds to an upper direction of the picture image.

11. The system according to claim 10,
wherein an indicated direction of the indicator and an indicated direction of the gravity indicator are recognizable by a user's sense of touch.

12. The system according to claim 1,
wherein the processing section is an image processing section which rotates a picture image of the specimen by image processing.

13. The system according to claim 1,
wherein the processing section rotates a picture image of the specimen by rotation of the imaging section.

14. A system comprising:
an inserting section having an insertion axis;
an imaging section which is provided to the inserting section, and takes an image of a specimen on a lateral side to the insertion axis;
an image generating section which generates a picture image of the specimen on the basis of an imaging signal output by the imaging section;
an operating section which is provided on a proximal end side of the inserting section, and operated by an operator;
a gravity direction indicating section which is provided on an opposite side of the lateral side of the operating section, and used by the operator to indicate a gravity direction; and
an image processing section which rotates the picture image of the specimen in correspondence with an indication of the gravity direction indicating section.
